(19) United States
(12) Patent Application Publication
(10) Pub. No.: US 2009/0053267 A1
(43) Pub. Date: Feb. 26, 2009

(54) DENTIFRICE COMPOSITION CONTAINING SILICONE OIL BEADS

(76) Inventors: Karen DePierro, Piscataway, NJ (US); Ralph Peter Santarpia, III, Edison, NJ (US); John Pierre Curtis, Alpha, NJ (US); Hollandra Niles, Somerset, NJ (US); Joann Darcy-Siegel, Somerset, NJ (US); Bernie L. Blackwell, Ringoes, NJ (US)

Correspondence Address: COLGATE-PALMOLIVE COMPANY, 909 RIVER ROAD, PISCATAWAY, NJ, 08855, US

(21) Appl. No.: 11/841,183

(22) Filed: Aug. 20, 2007

(51) Int. Cl. A61K 8/02; A61K 8/25; A61K 8/64; A61K 8/21; A61Q 11/00; A61K 8/66

(52) U.S. Cl. 424/401; 424/49; 424/50; 424/52

(57) ABSTRACT

Dentifrice composition and method of using such dentifrice composition to ameliorate dry mouth, where the dentifrice composition includes an orally acceptable vehicle having distributed therein silicone oil beads, and wherein the dentifrice composition has a viscosity of at least about 20,000 centipoise.

### BACKGROUND OF THE INVENTION

An effective dentifrice composition should provide maintenance and preservation of tooth appearance through the thorough cleaning of teeth and gums, removal of dental stains, and the polishing of the teeth. It should clean and remove exogenous debris, thereby aiding the prevention of tooth decay and promoting gingival health as well as providing clean and fresh mouth feel. However, conventional dentifrice compositions tend to be drying due to silicas and/or surfactants. As a result, it is desirable for a dentifrice composition to provide clean mouth feel and freshened breath while ameliorating the perception of dry mouth, such as that associated with xerostomia, and to provide positive mouth and/or tooth feel benefits.

### BRIEF SUMMARY OF THE INVENTION

A dentifrice composition includes an orally acceptable vehicle having distributed therein silicone oil beads, wherein the dentifrice composition has a viscosity of at least about 20,000 centipoise.

A method of ameliorating a perception of dry mouth includes applying a dentifrice composition to the oral cavity of a user, wherein the dentifrice composition comprises an orally acceptable vehicle having distributed therein silicone oil beads, and wherein the dentifrice composition has a viscosity of at least about 20,000 centipoise.

### DETAILED DESCRIPTION OF THE INVENTION

As used throughout, ranges are used as a shorthand for describing each and every value that is within the range. Any value within the range can be selected as the terminus of the range. Individuals on various types of medications or who suffer from certain medical conditions, such as radiation exposure or other injury to the head and neck, mumps, sarcoidosis; or ??? advancing age may experience a perception of “dry mouth”, i.e., the perception that insufficient saliva is present in the oral cavity. The compositions and methods of this invention prevent and/or ameliorate this perception.

The term “dentifrice” shall include toothpastes and gels and any other forms. The dentifrice composition may be in any desired from, such as deep striped, surface striped, multilayered, or any combination of gel and toothpaste.

The term “orally-acceptable vehicle” as used herein shall include a suitable vehicle, which can be used to apply the present compositions to the oral cavity in a safe and effective manner. Such vehicle may include, but is not limited to, materials such as fluoride ion sources, additional anticalculus agents, buffers, other abrasive materials, peroxide sources, alkali metal bicarbonate salts, thickening materials, humectants, water, surfactants, titanium dioxide, flavor system, sweetening agents, xylitol, coloring agents, and mixtures thereof.

In one embodiment, a dentifrice composition may include (1) a base dentifrice which may include an orally acceptable vehicle, and (2) silicone oil beads distributed throughout the orally acceptable vehicle. In other embodiments, a dentifrice composition may further include one or more active agents. A dentifrice composition including an orally acceptable vehicle with silicone oil beads distributed throughout the orally acceptable vehicle may be applied to the oral cavity of a user, e.g. a person, in order to ameliorate a perception of dry mouth. Each of the above described constituents and other constituents for use in various embodiments of the present invention are described hereafter.

A vehicle for the components of the dentifrice composition may include any orally-acceptable vehicle suitable for use in the oral cavity. Such carriers may include any of the usual components of toothpastes, tooth powders, prophylaxis pastes, lozenges, gums and the like, known or developed in the art.

In some embodiments, an orally acceptable vehicle includes a water-phase. In some embodiments, the dentifrice composition includes water in an amount of about 5% to about 10% by weight.

The orally acceptable vehicle may also include a humectant. In some embodiments, the humectant includes glycerine or sorbitol or an alkylene glycol such as polyethylene glycol or propylene glycol. In some embodiments, the humectant may be present in an amount of about 30% to about 80% by weight of the dentifrice composition. In other embodiments, the humectant may be present in an amount of about 50% to about 70% by weight.

The orally acceptable vehicle may include an inorganic or a natural or synthetic thickener or gelling agent. The thickener or gelling agent may be present in an amount of about 0.10% to about 5% by weight. The thickener or gelling agent may be present in an amount of about 0.2% to about 1% by weight. A suitable amount of thickener is included in the dentifrice composition to suspend the silicone oil beads. It may be desired that the thickener is included in the dentifrice composition to form an extrudable, shape-retaining product that can be squeezed from a tube onto a toothbrush and will not fall between the bristles of the brush but rather, will substantially maintain its shape thereon. Suitable thickeners or gelling agents for a dentifrice composition may include inorganic thickening silicas such as amorphous silicas available from Huber Corporation under the trade designation Zeodent 165, Irish moss, carrageenan, gum tragacanth, and polyvinylpyrrolidone.

In some embodiments, the orally acceptable vehicle includes at least one surfactant. Suitable surfactants may include, but are not limited to, water-soluble salts of higher fatty acid monoglyceride monosulfates, such as the sodium salt of the monsulfated monoglyceride of hydrogenated coconut oil fatty acids, cocamidopropyl betaine, higher alkyl sulfates such as sodium lauryl sulfate, alkyl aryl sulfonates such as sodium dodecyl benzene sulfonate, higher alkyl sulfoacetates, sodium lauryl sulfoacetate, higher fatty acid esters of 1,2-dihydroxy propane sulfonate, and the substantially saturated higher aliphatic acyl amities of lower aliphatic amino carboxylic acid compounds, such as those having 12 to 16 carbons in the fatty acid, alkyl or acyl radicals, and the like. Examples of the last mentioned amides are N-lauroyl sarcosine, and the sodium, potassium, and ethanolamine salts of N-lauroyl, N-myristoyl, or N-palmitoyl sarcosine.

In some embodiments, the anionic surfactants are present in the dentifrice composition in an amount of about 0.3% to about 5% by weight. In other embodiments, the anionic surfactants are present in the dentifrice composition in an amount of about 0.5% to about 2% by weight.

The dentifrice composition may also contain a binder agent. Examples of suitable binder agents may include, but are not limited to, marine colloids, carboxyvinyl polymers, carrageenans, starches, cellulosic polymers such as hydroxyethylcellulose, carboxymethylcellulose (carmellose), hydroxypropyl methyl cellulose and salts thereof (e.g., carmellose sodium), natural gums such as karaya, xanthan, gum arabic and tragacanth, chitosan, colloidal magnesium aluminum silicate, and colloidal silica.

In some embodiments, abrasives are added to the dentifrice formulation. Examples of suitable abrasives include, but are not limited to, silica abrasives such as precipitated silicas, sodium metaphosphate, potassium metaphosphate, tricalcium phosphate, dihydrated dicalcium phosphate, aluminum silicate, calcined alumina, bentonite or other siliceous materials, particulate thermosetting resins, such as melamine, phenolic, and urea-formaldehydes, and cross-linked polyepoxides and polyesters.

The dentifrice compositions may also include a flavorant or a mixture of flavorants, including natural or synthetic flavorants, such as flavoring oils, flavoring aldehydes, esters, alcohols, similar materials, and combinations thereof. Examples of suitable flavorants may include vanillin, spearmint oil, cinnamon oil, oil of wintergreen (methylsalicylate), peppermint oil, clove oil, anise oil, eucalyptus oil, citrus oils, fruit oils and essences. In some embodiments, the dentifrice composition includes flavorants such as limonene, menthone, carvone, menthol, anethole, eucalyptus oil, eucalyptol, anethole, eugenol, cassia, oxanone, .alpha.-irisone, propenyl guaiethol, thymol, linalool, benzaldehyde, cinnamaldehyde, N-ethyl-p-menthan-3-carboxamine, N,2,3-trimethyl-2-isopropylbutanamide, 3-1-menthoxypropane-1,2-diol, cinnamaldehyde glycerol acetal (CGA), methone glycerol acetal (MGA) and cineole.

Other additives may be included in the dentifrice composition for reasons of manufacturing, stability, aesthetics, therapeutic effect, consumer appeal, etc. Exemplary additives include all other conventional dentifrice additives, viscosity modifiers, diluents, foam modulators, saliva stimulating agents, desensitizing agents, whitening agents, enzymes, pH modifying agents, mouth-feel agents, sweeteners, colorants, opacifiers, and breath freshening agents.

The dentifrice composition may also include silicone oil beads. In some embodiments, the silicone oil beads are distributed throughout an orally acceptable vehicle.

Any suitable silicone oil can be included in the dentifrice composition. In some embodiments, the silicone oil is at least one of trisiloxane, cyclomethicone, dimethicone, phenyltrimethicone, or combinations thereof.

The silicone oil beads may be present in any suitable amount. In some embodiments, the silicone oil beads are present in an amount of about 1% to about 3% by weight or of about 5% to about 50% by weight of the dentifrice composition. In other embodiments, the silicone oil beads are present in an amount of about 20% to about 30% by weight of the composition.

The silicone beads included in the invention may be any known or developed in the art; such beads should, however, be capable of releasing the silicone oil into the oral cavity upon application. Such release may be effected by any means or mode, including instantaneous or substantially instantaneous release, continuous release, mechanical means, and/or chemical means (such as dissolution). The beads may be forms of, for example, polymers, such as celluloses, gelatin, starches, gums, peptides or proteins, lactose and other saccharide polymers.

The beads may be of any size. It may be preferred, however, that the beads of an average particle size of about 0.1 mm to about 1 mm or about 0.3 mm to about 0.5 mm or about 1 mm to about 3.5 mm, or up to about 5 mm.

A dentifrice composition may also include at least one active agent. The active agent may be within the beads (encapsulated with the silicone oil(s)); it may be within the carrier/vehicle; it may be embedded wholly or partially within the coating or outer shell of the beads.

Examples of suitable active agents include, but are not limited to, steroids, NSAIDs, a fluoride ion source, polycarboxylate polymers, polyvinyl methyl ether/maleic anhydride (PVME/MA) copolymers, an arginine ester, a zinc ion source, a stannous ion source, delmopinol, tartar control agents, an antibacterial agent, triclosan and salts thereof, chlorhexidine, alexidine, hexetidine, sanguinarine, benzalkonium chloride, salicylanilide, domiphenl bromide, cetylpyridinium chloride (CPC), tetradecylpyridinium chloride (TPC), N-tetradecyl-4-ethylpyridinium chloride (TDEPC), octenidine, octapinol, nisin, a zinc ion source, a copper ion source, an essential oil, a furanone, anti-inflammatory agents, antiplaque agents, antioxidants, and a bacteriocins, and salts thereof, honokiol, vitamins, anti-attachment agents, proteinaceous agents, peptides.

The composition of the invention may contain an anticaries active agent, such as fluoride ion providing compound, sodium fluoride, monosodium fluoride, calcium fluoride, and arginine salts, arginine bicarbonate, arginine phosphate, and arginine bicarbonate.

In some embodiments, the dentifrice composition is a paste or gel. In some embodiments, the constituents of the dentifrice composition are chosen such that the composition may have viscosity of at least about 10,000 centipoise.

A suitable amount of thickener may be included in the dentifrice composition to suspend the silicone oil beads. In some embodiments, a suitable amount of thickener is included in the dentifrice composition to form an extrudable, shape-retaining product which can be squeezed from a tube onto a toothbrush and will not fall between the bristles of the brush but rather, will substantially maintain its shape thereon.

A dentifrice composition as described above may be applied to an oral cavity. In some embodiments, application of the dentifrice composition to an oral cavity ameliorates dry mouth feel, such as that associated with xerostomia.

In some embodiments, application of the dentifrice composition to an oral cavity provides a clean yet moist feel in the oral cavity. In some embodiments, application of the dentifrice composition to an oral cavity results in positive mouth and/or tooth feel. In some embodiments, positive mouth and/or tooth feel includes at least one of the following qualities: superior clean feeling, fresh feeling, shiny tooth appearance, smooth tooth feel, cool mouth feel, fresh breath, moist mouth, hydrated mouth, and extended or lasting fresh breath.

In some embodiments, the dentifrice composition is a toothpaste and/or gel. In some embodiments, the dentifrice composition is applied directly to the teeth, such as by a toothbrush, by hand, by swab, or by a film or strip.

### EXAMPLE

A dentifrice composition according is prepared by combining the following ingredients.

| Ingredient | Weight Percent |
|---|---|
| Sorbitol | 60 |
| Polyethylene glycol 600 | 3.0 |
| Sodium CMC 7MF | .5 |
| Tetrasodium | 0.5 |
| Pyrophosphate | |
| Deionized water | 6 |
| Sodium Saccharin | 0.2 |
| Sodium Fluoride | 0.2 |
| Beads with silicone oil | 2.5 |
| Abrasive silica | 25 |
| Color | 0.1 |
| Flavor | 1 |
| Sodium Lauryl Sulfate | 1 |
| | 100 |

What is claimed is:

1. A dentifrice composition comprising
an orally acceptable vehicle comprising one or more silicone oil beads,
wherein the dentifrice composition has a viscosity of at least about 20,000 centipoise.

2. The dentifrice composition of claim 1, wherein the silicone oil comprises at least one member chosen from trisiloxane, dimethicone, simethicone, phenyltrimethicone, and cyclomethicone.

3. The dentifrice composition of claim 1, further comprising an active agent.

4. The dentifrice composition of claim 1, further comprising at least one agent chosen from abrasives, silica, pyridinium chlorochromate (PCC), chlorhexidine, alexidine, hexetidine, and sanguinarine.

5. The dentifrice composition of claim 1, further comprising at least one agent chosen from benzalkonium chloride, salicylanilide, domiphen bromide, cetylpyridinium chloride (CPC), tetradecylpyridinium chloride (TPC) and N-tetradecyl-4-ethylpyridinium chloride (TDEPC).

6. The dentifrice composition of claim 1, further comprising at least one agent chosen from octenidine, octapinol, nisin, a zinc ion source, a copper ion source, an essential oil, and a furanone.

7. The dentifrice composition of claim 1, further comprising at least one agent chosen from an enzyme, a peptide, a protein, a bacteriocin and salts thereof, and honokiol.

8. The dentifrice composition of claim 1, further comprising at least one member chosen from humectant, flavorant, and surfactant.

9. A method of ameliorating dry mouth comprising applying a dentifrice composition to the oral cavity of a user, wherein the dentifrice composition comprises an orally acceptable vehicle having distributed therein silicone oil beads, and wherein the dentifrice composition has a viscosity of at least about 10,000 centipoise.

10. The method of claim 9, wherein the silicone oil comprises at least one member chosen from trisiloxane, dimethicone, simethicone, phenyltrimethicone, and cyclomethicone.

11. The method of claim 9, wherein the dentifrice composition further comprises an active agent.

12. The method of claim 9, wherein the composition further comprises at least one member chosen from abrasives, silica, pyridinium chlorochromate (PCC), chlorhexidine, alexidine, hexetidine, and sanguinarine.

13. The method of claim 9, wherein the composition further comprises at least one member chosen from benzalkonium chloride, salicyanilide, domiphen bromide, cetylpyridinium chloride (CPC), tetradecylpyridinium chloride (TPC), and N-tetradecyl-4-ethylpyridinium chloride (TDEPC).

14. The method of claim 9, wherein the composition further comprises at least one member chosen from octenidine, octapinol, nisin, a zinc ion source, a copper ion source, an essential oil, a furanone.

15. The method of claim 9, wherein the composition further comprises at least one member chosen from an enzyme, a peptide, a protein, a bacteriocin and salts thereof and honokiol.

16. The method of claim 9, wherein the dentifrice composition further comprises at least one member chosen from a humectant, a flavorant, and a surfactant.

17. The method of claim 9, wherein the dentifrice composition is applied directly to teeth of a user.

18. A method of preventing and/or reducing caries in an individual suffering from dry mouth comprising: applying a dentifrice composition to the oral cavity of a user, wherein the dentifrice composition comprises an orally acceptable vehicle having distributed therein silicone oil beads, has a viscosity of at least about 20,000 centipoise, and the composition contains an anticaries agent.

19. The method of claim 18, wherein the anticaries agent in present in the composition at a site chosen from at least one of inside the silicone oil beads, outside the silicone oil beads, wholly embedded in the silicone oil bead shell, and partially embedded within the silicone oil bead shell.

20. The method of claim 18, wherein the anticaries agent is chosen from a fluoride ion providing compound, sodium fluoride, monosodium fluoride, calcium fluoride, and arginine salts.

21. The method of claim 18, wherein the anticaries agent is selected from arginine bicarbonate, arginine phosphate, and arginine bicarbonate.
